(19) United States
(12) Patent Application Publication
(10) Pub. No.: US 2014/0058180 A1
(43) Pub. Date: Feb. 27, 2014

(54) MODIFIED CATALYST FOR CONVERTING OXYGENATES TO OLEFINS

(71) Applicants: Stefan Klingelhöfer, Rosenheim (DE); Manfred Frauenrath, Grosskarolinenfeld (DE); Gotz Burgfels, Bad Abiling (DE)

(72) Inventors: Stefan Klingelhöfer, Rosenheim (DE); Manfred Frauenrath, Grosskarolinenfeld (DE); Gotz Burgfels, Bad Abiling (DE)

(73) Assignee: CLARIANT PRODUKTE (DEUTSCHLAND) GmbH, Frankfurt/Main (DE)

(21) Appl. No.: 14/005,315

(22) PCT Filed: Mar. 15, 2012
(86) PCT No.: PCT/EP2012/054613

§ 371 Date: Nov. 13, 2013

(30) Foreign Application Priority Data
Mar. 15, 2011 (DE) 10 2011 013 908.7

(51) Int. Cl. B01J 29/40; C07C 1/22

(52) U.S. Cl. CPC B01J 29/40; C07C 1/22
USPC 585/640; 502/64; 502/60

(57) ABSTRACT

The present invention relates to a new process for producing zeolite-containing catalysts, in which a modification with phosphorus-containing components is carried out, the catalyst obtainable thereby, and its use as catalyst in a process for producing lower olefins from oxygenates. The modification comprises removing weakly bound phosphorus-containing species by treatment with an aqueous solution.

The present invention relates to a process for producing zeolite-based phosphorus-containing catalysts and use thereof in a process for producing lower olefins from oxygenates. The process is useful in particular for increasing the methanol conversion rate in this process.

### BACKGROUND OF THE INVENTION

The conversion of oxygenates (oxygen-containing compounds), such as methanol and/or dimethyl ether, to olefins, in particular propylene, has long been known. The production of propylene is of considerable economic interest, as propylene is an important raw material for obtaining polypropylene, which among other things is used in machine and vehicle construction and in electrical engineering. The propylene obtained by conversion from methanol is preferable to the propylene obtained by thermal cracking of hydrocarbons, as it is practically free from sulphur compounds. Crystalline aluminosilicates are often used as catalysts in this conversion process.

A process of this kind is known from U.S. Pat. No. 4,058,576. In a first stage, methanol is converted using an acid catalyst, such as gamma-aluminium oxide, in an exothermic condensation reaction at least partially to dimethyl ether. In this way, part of the reaction heat of the conversion of methanol to lower olefins taking place in the second step can be removed, as the heat produced in the exothermic reaction when using dimethyl ether as starting material is less than when using methanol. In the second stage the reaction takes place via a crystalline zeolite of the ZSM-5 type. This is a crystalline aluminosilicate of the pentasil type, which is preferably to have a ratio of silica to alumina of at least 12 and a pore size greater than 0.5 nm.

The reaction in the second stage takes place in a tubular reactor, obtaining, as lower olefins, preferably those with three or more carbon atoms (C3+ olefins). These lower olefins are then converted using the ZSM-5 catalyst at increased pressure to hydrocarbons in the light gasoline boiling range.

EP 0 369 364 A2 describes a catalyst based on crystalline aluminosilicates of the pentasil type in H-form, which is made up of primary crystallites with an average diameter of from 0.1 to 0.9 μm, which are combined to at least 20% into agglomerates of from 5 to 500 μm, wherein the catalyst contains finely-divided aluminium oxide as binder in an amount from 10 to 40 wt.-%. The catalyst has a BET surface area of from 300 to 600 m²/g and a pore volume of from 0.3 to 0.8 cm³/g and is intended for application in a CMO (Conversion of Methanol to Olefins) process. The selectivity for C2-C4 olefins is 50 to 55 wt.-%.

A general problem when using catalysts in the conversion of oxygenates to olefins is the catalyst's tendency to lose catalytic activity in the course of the process. This is caused on the one hand by the increasing coking of the surfaces and pores. This arises because the by-products that form during conversion of the oxygenates to olefins condense to longer-chain or ring-shaped species and can be deposited on the catalyst, so that the catalytically active sites are masked. Therefore after a certain operating time, a so-called regeneration is required, in which the carbon-containing deposits are removed from the catalyst in mild conditions. On the other hand, the reaction conditions also lead to a progressive dealumination of the zeolitic material. This is caused by the steam that forms during conversion of the oxygenates. Dealumination leads to a gradual decrease in the number of catalytically active sites, the catalyst is deactivated irreversibly and the conversion rate of the oxygenate used decreases.

The modification of zeolites with phosphorus-containing components is known from the literature. The respective phosphorus-containing species are applied in particular by impregnation, ion exchange, CVD processes and pore-filling strategies. The catalysts produced in this way are characterized in particular by improved catalytic properties in alkylation reactions, in cracking processes and in the conversion of oxygenates to olefins.

Lischke et al. (Journal of Catalysis, 132, (1991), 229-243) describe the treatment of strongly acidic zeolites of the ZSM-5 type with phosphoric acid solution and in an impregnation process. The samples loaded in this way are then dried or calcined in a steam atmosphere at various temperatures. By a subsequent washing procedure with hot water, variable amounts of phosphate are removed from the material again. The zeolite used in this publication is characterized by a very low silicon-aluminium ratio and is therefore unsuitable for the conversion of oxygenates to olefins, as in this case too many undesired by-products would form.

EP 2 025 402 A1 discloses the use of a phosphorus-containing zeolite in the conversion of methanol to olefins. The catalysts are produced by steam treatment of a zeolite with an Si/Al ratio of below 1:30 at a temperature in the range of from 550 to 680° C.; washing out of a proportion of the Al from the zeolite with an aqueous phosphorus-containing solution; separation of the zeolite from the liquid and calcining of the zeolite.

WO 2006/127827 A2 relates to a process for producing zeolite catalysts comprising: treating a zeolite with a phosphorus compound to form a phosphorus-treated zeolite; heating the phosphorus-treated zeolite to a temperature of about 300° C. or higher; reacting the phosphorus-treated zeolite with an inorganic oxide binder to form a zeolite-binder mixture and heating the zeolite-binder mixture to a temperature of 400° C. or more. These catalysts are used for the alkylation of aromatic compounds, in particular for the methylation of toluene.

Therefore there is still a need for a catalyst that has improved stability in a process for producing olefins from oxygenates, and in particular shows an improved conversion rate of the oxygenate used. This problem is solved by the process according to the invention and the catalysts obtainable therewith.

### DESCRIPTION OF THE INVENTION

The invention relates to a process for producing a phosphorus-containing catalyst, comprising the following steps:
(a) applying a phosphorus-containing compound to a zeolite,
(b) calcining the modified zeolite,
(c) treating the calcined zeolite from step (b) with an aqueous solution or water, in order to remove a proportion, in particular at least 50 wt.-%, preferably at least 70 wt.-%, particularly preferably 80 to 95 wt.-% of the phosphorus-containing component, optionally carrying out another calcination,
(d) mixing the material from step (c) with a binder,
(e) shaping the binder-zeolite mixture from step (d), and
(f) calcining the shaped material from step (e).

It was found, surprisingly, that the catalysts obtained with the process according to the invention have, in the production of lower olefins from oxygenates, in particular from methanol and/or dimethyl ether, an improved conversion rate of the oxygenate.

The invention therefore also relates to the phosphorus-containing catalyst obtainable with the process according to the invention, and use thereof for converting oxygenates, in particular methanol, dimethyl ether and/or mixtures thereof, to olefins.

Advantageously, in the present invention, treatment of the zeolite with steam is not used during calcining, to prevent dealumination of the zeolite taking place and thus altering the material. Particularly preferably, in step (b) and/or in step (f), quite particularly preferably in step (b) and in step (f), treatment of the zeolite with steam is not used during calcining. In contrast, through the process according to the invention, the amount of phosphate is adjusted by treatment with water or aqueous solution in step (c), which leads to increased stability of the catalyst obtained.

In the process according to the invention, after step (a) the catalyst contains a considerable quantity of phosphate species, although the zeolite used is characterized by a relatively low concentration of Brønsted acid sites. The purely thermal treatment in the absence of significant amounts of steam appears to give particularly advantageous interaction between zeolite structure and applied phosphate species, and this seems to be responsible for the improved stability.

The zeolite used in step (a) is usually a crystalline aluminosilicate zeolite. The zeolite can have a structure as described in the “Atlas of Zeolite Framework Types” (Ch. Baerlocher, W. M. Meier, D. H. Olson, Elsevier, Fifth Revised Edition, 2001), whose disclosure in this respect is hereby incorporated in the description. Suitable zeolite materials are for example zeolites with the TON structure (e.g. ZSM-22, ISI-1, KZ-2), MTT structure (e.g. ZSM-23, KZ-1), MFI structure (e.g. ZSM-5), MEL structure (e.g. ZSM-11), MTW structure (e.g. ZSM-12), zeolites with the EUO structure or also ZSM-21, ZSM-35, ZSM-38, ZSM-4, ZSM-18 or ZSM-57. In particular the zeolite has a TON structure, MTT structure, MFI structure, MEL structure, MTW structure or EUO structure. Mixtures of zeolites of different structure can also be used. Preferably the zeolite used in step (a) is a zeolite of the pentasil type; particularly preferably the zeolite has an MFI structure, in particular of the ZSM-5 type. It is furthermore preferable for the zeolites to be present in the H-form, i.e. the protonated form.

The pores present in the zeolite material used preferably have radii of from 4.0 Å to 6.0 Å, particularly preferably of from 4.8 Å to 5.8 Å.

The zeolite powder used in the process according to the invention is in addition preferably obtained by adding a template to the synthesis gel. Tetraalkylammonium compounds, preferably tetrapropylammonium hydroxide (TPAOH) or tetrapropylammonium bromide (TPABr), are used as templates. Mixtures of ammonia or an organic amine and another organic compound from the group of the alcohols, preferably butanol, can also be used as templates.

The zeolite used in step (a) preferably has an Si/Al atomic ratio in the range of from 50 to 250, preferably in the range of from 50 to 150, in particular in the range of from 75 to 120, still more preferably in the range of from 85 to 110.

The phosphorus-containing compound can be used as a solid or in solution, preferably in aqueous solution. It is preferable for the phosphorus-containing compound to be used in solution. If the phosphorus-containing compound is applied in step (a) to the zeolites in the form of a solution, the product obtained is usually dried before it is subjected to the calcination step (b). In step (a), the phosphorus-containing compound is preferably applied to the zeolite by spray-drying. This is usually carried out by first suspending the zeolite in the phosphorus-containing solution, optionally heating the suspension for improved interaction of the phosphorus-containing component with the zeolite and then spray-drying.

In the process according to the invention, the phosphorus-containing compound is preferably selected from inorganic phosphorus-containing acids, organic phosphorus-containing acids, alkali, alkaline-earth and/or ammonium salts of inorganic phosphorus-containing acids or organic phosphorus-containing acids, phosphorus(V) halides, phosphorus(III) halides, phosphorus oxide halides, phosphorus(V) oxide, phosphorus(III) oxide and mixtures thereof.

In the process according to the invention it is moreover preferable for the phosphorus-containing compound to be selected from PY₅, PY₃, POY₃, MₓE_{z/2}H_{3-(x+z)}PO₄, MₓE_{z/2}H_{3-(x+z)}PO₃, P₂O₅ and P₄O₆,

in which Y denotes F, Cl, Br or I, preferably Cl,
x=0, 1, 2 or 3,
z=0, 1, 2, or 3,
with x+z≦3,
M denotes independently alkali metal and/or ammonium, and
E denotes alkaline-earth metal.

In an even more preferred embodiment, the phosphorus-containing compound used in the process according to the invention is H₃PO₄, (NH₄) H₂PO₄, (NH₄)₂HPO₄ and/or (NH₄)₃PO₄. In the process according to the invention it is particularly preferable that the phosphorus-containing compound is H₃PO₄.

In the process according to the invention, a calcining is carried out usually for 10 min to 15 h, preferably for 1 h to 12 h. The calcining temperature is usually 150° C. to 800° C., preferably 300° C. to 600° C. It is particularly preferable for the calcining in step (b) to be carried out for 5 h to 15 h, in particular for 10 h, at a temperature in the range of from 400° C. to 700° C., in particular at 500° C. to 600° C., particularly preferably at about 540° C. It is more preferable for the calcining in step (f) to be carried out for 5 h to 15 h, in particular for 10 h, at a temperature in the range of from 400° C. to 700° C., in particular at 500 to 600° C., particularly preferably at about 540° C.

The binder used in step (d) in the process according to the invention is usually inorganic oxides, in particular aluminium oxide, magnesium oxide, titanium oxide, zinc oxide, niobium oxide, zirconium oxide, silicon oxide, and/or hydrates thereof, and mixtures thereof, e.g. mixtures of the aforementioned oxides (except aluminium oxide) with aluminium oxide. For example, amorphous aluminosilicates and non-oxidic binder such as aluminium phosphates for example can also be used as binder in step (d). The binder used in step (d) is preferably an aluminium oxide, which can also be used as hydrated aluminium oxide or as modified aluminium oxide. Modified aluminium oxide is for example phosphorus-modified aluminium oxide. It is particularly preferable to use finely-divided aluminium oxide, which is obtained for example by hydrolysis of aluminium trialkyls or aluminium alcoholates, or is used in the form of peptizable hydrated aluminium oxide. Quite particularly preferably, peptizable hydrated aluminium oxide is used as binder. Preferably at least 95% of the particles of the peptizable hydrated aluminium oxide have an average diameter of ≦100 μm, measured by laser diffraction. A MALVERN MasterSizer 2000 with 2000 S dispersing unit was used for the determination; measurement was carried out according to ISO 13320.

Mixing of the material from step (c) with a binder in step (d) is usually carried out by means of a commercially available mixer, e.g. a mixer with moving mixing tools and fixed chamber or a mixer with moving mixing tools and moving chamber.

It is preferable to use the binder in an amount from 5 to 60 wt.-%, more preferably 10 to 40 wt.-%, particularly preferably 15 to 35 wt.-%, relative to the total weight of zeolite used and binder.

The aqueous solution or water used in step (c) is preferably selected from water, aqueous ammonium chloride, dilute hydrochloric acid, dilute acetic acid and dilute nitric acid. It is preferable to use water in step (c). The aqueous solution or water used in step (c) serves for removing a proportion of the phosphorus-containing compound applied in step (a).

Preferably the calcined zeolite obtained in step (b) is treated with the aqueous solution or water until at least 50 wt.-%, in particular at least 70 wt.-%, particularly preferably 80 to 95 wt.-% of the phosphorus-containing compound has been removed. The duration and amount and optionally concentration of the aqueous solution or water can easily be determined by a person skilled in the art. For example the calcined zeolite is slurried with water for about 30 min to 3 h at 80 to 90° C. and the powder is separated from the liquid medium after the treatment. Usually after the treatment of the calcined zeolite with an aqueous solution or water, in step (c) the zeolite is filtered off, washed with water, dried and calcined again, before the material is mixed with the binder in step (d).

In step (e), the binder-zeolite mixture from step (d) undergoes a shaping. Shaping usually means in the present invention the transforming of a material into a shaped body with defined dimensions. The shaped bodies obtainable by shaping include for example extrudates, spheres, honeycombs, pellets, and granules. The shaping in step (e) can be carried out for example using a commercially available extruder, e.g. a single-screw extruder or twin-screw extruder. In particular, the shaping in step (e) can start with a plasticizable material, which, on completion of shaping, then undergoes calcining in step (f), to obtain the desired stability.

The catalyst obtainable by the process according to the invention preferably has a BET surface area in the range of from 300 to 500 m²/g, in particular from 310 to 450 m²/g and particularly preferably from 320 to 400 m²/g, determined according to DIN 66131.

The catalyst according to the invention is further characterized by an Na content of preferably less than 200 ppm, in particular less than 150 ppm.

The pore volume of the catalyst according to the invention, determined by the mercury porosimetry method according to DIN 66133, is preferably 0.3 to 0.8 cm³/g, in particular 0.30 to 0.35 cm³/g.

The catalyst according to the invention can be used particularly advantageously in processes for producing olefins by conversion of oxygenates.

The catalyst according to the invention can therefore also be used particularly advantageously in processes for producing olefins by conversion of oxygenates, as the zeolite material used in the process according to the invention has an Si/Al atomic ratio that is in the range of from 50 to 250, preferably in the range of from 50 to 150, in particular in the range of from 75 to 120, more preferably in the range of from 85 to 110.

In principle, however, use in other carbon conversion reactions, such as in particular dewaxing processes, alkylations, conversion of paraffin to aromatic compounds (CPA) and related reactions is possible.

Therefore a process for producing olefins from oxygenates, preferably from methanol, dimethyl ether or mixtures thereof, wherein an educt gas, i.e. the gaseous starting material, is passed over the catalyst according to the invention, forms part of the invention. Oxygenates are to be understood, in the context of the present invention, as oxygen compounds, in particular organic oxygen compounds such as alcohols and ethers. The present invention therefore preferably relates to a process for producing lower olefins, in particular C₂ to C₆ olefins, from oxygen compounds (Oxygenates to Olefins, OTO), preferably from alcohols and/or ethers, particularly preferably from methanol (Methanol to Olefins, MTO) and/or dimethyl ether by reacting for example a reaction mixture containing methanol and/or dimethyl ether vapour and steam in a reactor using an indirectly cooled catalyst according to the invention. According to the process of the invention, in particular the methanol conversion in a reaction cycle is increased.

The conversion with the catalyst according to the invention preferably takes place (a) at a total pressure of from 10 to 150 kPa, in particular at a total pressure of from 50 to 140 kPa, (b) at a weight ratio of water to methanol or methanol equivalent of from 0.1 to 4.0, in particular of from 0.5 to 3, and (c) at a temperature of the reactor coolant of from 280 to 570° C., preferably of from 400 to 550° C. Such a process is described in EP 0 448 000 A1, whose disclosure in this respect is hereby incorporated in the description. Other preferred processes are described in EP 1 289 912 A1 and DE 10 2006 026 103 A1, whose disclosure in this respect is hereby incorporated in the description.

The present invention will be explained by the following non-limiting examples.

### EXAMPLES

The particle size of the primary particles was determined by scanning electron microscopy using a LEO 1350 scanning electron microscope. For sample preparation, the material was suspended in acetone, treated for 30 s in an ultrasonic bath and then placed on a sample carrier. Then the diameter of a large enough number of particles (about 10 to 20) is determined at 80,000× magnification. The mean value of the measured diameters is designated as particle size.

The mean lateral compressive strength was determined from the force that acts on the lateral face (longest side) of the shaped body until fracture occurs. For this, 50 shaped bodies with a length in the range of from 5.5 to 6.5 mm were selected from a representative sample of shaped bodies and were measured individually. The shaped bodies were formed crack-free and straight. A shaped body was placed between two jaws (one moving jaw and one fixed jaw). The moving jaws were moved uniformly towards the shaped body, until fracture of the shaped body occurred. The measured value at fracture in kilopond (kp), measured with a measuring instrument from Schleuniger, was divided by the length of the shaped body, to obtain the lateral compressive strength (in kp/mm or N/mm) of the shaped body. The mean lateral compressive strength was then determined from 50 individual measurements.

### Example 1

### Production of Catalyst 1 According to the Invention

An H-form ZSM-5 material, which had an Si/Al ratio of 99:1 and a BET surface area of 427 m²/g, was used as the zeolite to be modified. The zeolite was produced as disclosed in EP 0 369 364 A1, synthesis being terminated as soon as the primary crystals had reached a particle size of about 0.03 μm.

1200 g of the zeolite material was suspended in 6050 g of a phosphoric acid solution (about 1.5 wt.-% in water) at 80° C. for 2 h. Then the suspension was concentrated to dryness by means of a spray-drying process. This step was carried out in a NIRO spray dryer; the suspension was introduced into the spray dryer via a nozzle at a temperature of approx. 220° C. The resultant finely-divided product was then separated in a cyclone. The powder obtained was then calcined for approx. 10 h at 540° C. The phosphorus content of this intermediate product was 2.3 wt.-%, and the BET surface area had decreased as a result of the treatment to a value of 327 m²/g.

In the next step, 800 g of the powder thus obtained was slurried in 4000 ml dist. H₂O and was stirred for 1 h at 90° C. Then the powder treated in this way was filtered off, washed, dried at 120° C. for 4 h and calcined at 540° C. for 10 h.

As a result, the phosphorus content had been able to be reduced to a value of 0.37 wt.-%, which corresponds to a reduction to approx. 16%. The BET surface area had increased to a value of 383 m²/g.

For shaping, 700 g of the modified powder was mixed with 181 g of hydrated aluminium oxide and 28 g of paraffin wax. Then 245 g dist. H₂O and 48.5 g of nitric acid solution (5 wt.-% HNO₃) were added to this mixture, followed by a further 102 g dist. H₂O, until a plasticizable material was obtained. This was then mixed with 56 g of steatite oil.

Shaping was carried out by means of a commercially available extruder, e.g. a single-screw extruder or twin-screw extruder. The resultant shaped bodies had a diameter of approx. 3 mm and a length of approx. 6 mm. The shaped bodies were dried at 120° C. for 16 h and were calcined at 550° C. for 5 h. The phosphorus content of catalyst 1 obtained was 0.31 wt.-%, the BET surface area was determined as 369 m²/g, and the pore volume was 0.34 cm³/g. Measurement of the lateral compressive strength gave a value of 1.05 kp/mm (10.3 N/mm).

### Example 2

### Production of Catalyst 2 According to the Invention

An H-form ZSM-5 material, which had an Si/Al ratio of 105:1 and a BET surface area of 434 m²/g, was used as the zeolite to be modified. The zeolite was produced as disclosed in EP 0 369 364 A1, the synthesis being terminated as soon as the primary crystals had reached a particle size of about 0.03 μm.

1400 g of the zeolite material was suspended in 7066 g of a phosphoric acid solution (about 0.8 wt.-% in water) at 80 to 90° C. for 2 h. Then the suspension was concentrated to dryness by means of a spray-drying process as described in Example 1. The powder obtained was then calcined for approx. 10 h at 540° C. The phosphorus content of the intermediate product thus obtained was 1.2 wt.-%, and the BET surface area had decreased as a result of the treatment to a value of 394 m²/g.

In the next step, 850 g of the intermediate product was slurried in 4130 ml dist. H₂O and was stirred for 1 h at 90° C. Then the treated powder was filtered off, washed and after drying at 120° C. was calcined again at 540° C. for 10 h. As a result, the phosphorus content had been able to be reduced to a value of 0.09 wt.-%, which corresponds to a reduction to approx. 8%. The BET surface area had increased to a value of 409 m²/g.

For shaping, 700 g of the modified powder was mixed with 176 g of hydrated aluminium oxide and 28 g of paraffin wax. Then 245 g dist. H₂O and 48.3 g of nitric acid solution (5 wt.-% HNO₃) were added to this mixture, followed by a further 120 g dist. H₂O, until a plasticizable material was obtained. This was then mixed with 56 g of steatite oil. Shaping was carried out by means of a commercially available extruder, and the resultant shaped bodies had a diameter of approx. 3 mm and a length of approx. 6 mm. The shaped bodies were dried at 120° C. and calcined at 550° C. for 5 h. The phosphorus content of catalyst 2 obtained was 0.09 wt.-%, the BET surface area was determined as 387 m²/g and the pore volume was 0.34 cm³/g. Measurement of the lateral compressive strength gave a value of 0.90 kp/mm (8.83 N/mm).

### Example 3

### Production of Comparative Catalyst 1

An H-form ZSM-5 material, which had an Si/Al ratio of 99:1 and a BET surface area of 427 m²/g, was used as the zeolite to be modified. The zeolite was produced as disclosed in EP 0 369 364 A1, the synthesis being terminated as soon as the primary crystals had reached a particle size of about 0.03 μm.

1200 g of the zeolite material was suspended in 6050 g of a phosphoric acid solution (about 1.5 wt.-% in water) at 80° C. for 2 h. Then the suspension was concentrated to dryness by means of a spray-drying process. This step was carried out in a NIRO spray dryer; the suspension was introduced into the spray dryer via a nozzle at a temperature of approx. 220° C. The resultant finely-divided product was then separated in a cyclone. The powder obtained was calcined for approx. 10 h at 540° C. The phosphorus content of this intermediate product was 2.3 wt.-%, and the BET surface area had decreased as a result of the treatment to a value of 327 m²/g.

For shaping, 700 g of the modified powder was mixed with 179 g of hydrated aluminium oxide and 28 g of paraffin wax. Then 245 g dist. H₂O and 48.0 g of nitric acid solution (5 wt.-% HNO₃) were added to this mixture, followed by a further 127 g dist. H₂O, until a plasticizable material was obtained. This was then mixed with 56 g of steatite oil.

Shaping was carried out by means of a commercially available extruder, and the resultant shaped bodies had a diameter of approx. 3 mm and a length of approx. 6 mm. The shaped bodies were dried at 120° C. and calcined at 550° C. for 5 h. The phosphorus content of the resultant comparative catalyst 1 was 2.00 wt.-%, the BET surface area was determined as 337 m²/g and the pore volume was 0.43 cm³/g. Measurement of the lateral compressive strength gave a value of approx. 0.14 kp/mm (1.37 N/mm).

### Example 4

### Production of Comparative Catalyst 2

An H-form ZSM-5 material, which had an Si/Al ratio of 86:1 and a BET surface area of 363 m²/g, was used as zeolite. The zeolite was produced as disclosed in EP 0 369 364 A1, the synthesis being terminated as soon as the primary crystals had reached a particle size of about 0.03 μm.

1200 g of the zeolite material was suspended in 4403 g of a phosphoric acid solution (about 2.1 wt.-% in water) at approx. 95° C. for 2 h. Then the suspension was concentrated to dryness by means of a spray-drying process as described in Example 1. The powder was then calcined for approx. 10 h at 540° C. The phosphorus content of the intermediate product was 2.1 wt.-%, and the BET surface area had a value of 292 m²/g.

For shaping, 147.1 g of hydrated aluminium oxide was slurried with 150.5 g dist. H₂O and mixed intimately by stirring with 183.3 g of nitric acid solution (31 wt.-% in water). Then 600 g of the intermediate product was added to the viscous material and was homogenized, kneading continuously. A plasticizable material was obtained, which was mixed with 50.4 g of steatite oil.

Shaping was carried out by means of a commercially available extruder, and the resultant shaped bodies had a diameter of approx. 3 mm and a length of approx. 6 mm. The shaped bodies were dried at 120° C. and were calcined at 600° C. for 5 h. The phosphorus content of the resultant comparative catalyst 2 was 1.88 wt.-%, the BET surface area was determined as 285 m²/g and the pore volume as 0.27 cm³/g. Measurement of the lateral compressive strength gave a value of approx. 2.50 kp/mm (24.52 N/mm).

### Example 5

### Production of Comparative Catalyst 3

An H-form ZSM-5 material, which had an Si/Al ratio of 86:1 and a BET surface area of 363 m²/g, was used as zeolite. The zeolite was produced as disclosed in EP 0 369 364 A1, the synthesis being terminated as soon as the primary crystals had reached a particle size of about 0.03 μm.

54.6 kg of hydrated aluminium oxide was slurried with 65.6 kg dist. H₂O and was mixed intimately by stirring with 48.4 kg of nitric acid solution (12.8 wt.-% in water). Then 220.0 kg of the zeolite powder was added to the viscous material and was homogenized, stirring continuously. 4.4 kg of paraffin wax was also added. A plasticizable material was obtained, which was mixed with 18.5 kg of steatite oil.

Shaping was carried out by means of a commercially available extruder, and the resultant shaped bodies had a diameter of approx. 3 mm and a length of approx. 6 mm. The shaped bodies were dried at 120° C. and calcined at 550° C. for 5 h. The BET surface area of the resultant comparative catalyst 3 was determined as 340 m²/g, and the pore volume was 0.37 cm³/g. Measurement of the lateral compressive strength gave a value of 1.09 kp/mm (10.69 N/mm).

Comparative catalyst 1 with a high phosphorus loading of about 2.0 wt.-% is insufficiently suitable for further processing to a shaped body, as its lateral compressive strength (approx. 0.14 kp/mm) is so low that there are problems here in transport and in filling the reactor, as the shaped bodies very quickly disintegrate. Therefore, for comparative catalyst 2 the shaping operation was modified, in order to increase the lateral compressive strength. However, this led to such a marked decrease in pore volume that it was not possible to use this catalyst in the CMO process. The marked decrease in BET surface area of about 100 m²/g to 292 m²/g also represented a marked impairment for use as catalyst in surface-active processes. It can be assumed that owing to excess phosphate species, an interaction occurs between the binder material used, which had already been attacked on the surface by the earlier addition of acid solution and was therefore more reactive, and the other components in the shaping operation, so that catalysts with markedly decreased total pore volumes and BET surface areas are obtained.

### Application Example 1

The catalyst according to the invention from Example 1 and comparative catalyst 3 were in each case filled in a vertical fixed-bed reactor and were treated with steam for 48 h. Then the reaction was started, wherein a reaction mixture consisting of methanol and steam was passed over the catalyst. The loading of the catalysts with methanol was 1/h, i.e. 1 g of methanol was passed over 1 gram of catalyst per hour. The temperature at reactor inlet was 450° C., and the test was carried out for 850 h, wherein 2 cycles were carried out. After the first cycle (after about 450 h), a regeneration was carried out, by first increasing the reactor temperature under nitrogen atmosphere to 480° C. and then progressively increasing the proportion of oxygen, until the composition corresponded to that of air. As soon as no further decomposition of carbon-containing components could be detected, regeneration was stopped and the reactor conditions were returned to those prevailing at the beginning of the 1st cycle.

Table 1 shows the conversion rates of catalyst 1 according to the invention and those of comparative catalyst 3 for different operating times tos (time on stream).

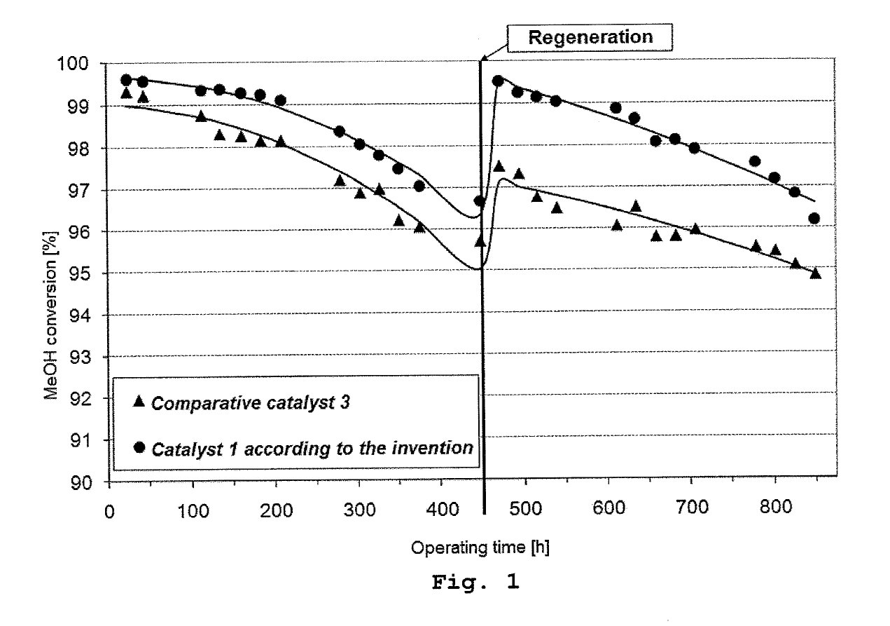

FIG. 1 shows a graph of methanol conversion as a function of the operating time.

TABLE 1
Methanol conversion as a function of operating time

| | MeOH conversion | MeOH conversion |
|---|---|---|
| tos [h] | Catalyst 1 [%] | Comparative catalyst 3 [%] |
| 24 | 99.61 | 99.33 |
| 114 | 99.35 | 98.76 |
| 185 | 99.23 | 98.15 |
| 280 | 98.36 | 97.19 |
| 328 | 97.78 | 96.98 |
| 376 | 97.04 | 96.05 |

Regeneration

| | | |
|---|---|---|
| 517 | 99.14 | 96.75 |
| 659 | 98.08 | 95.79 |
| 707 | 97.91 | 95.96 |
| 802 | 97.16 | 95.43 |

Over the total operating time of a total of 850 h, a methanol conversion of 98.3% is achieved for the catalyst according to the invention, but only a conversion of approx. 96.8% for comparative catalyst 3.

The excellent properties of the catalyst according to the invention are particularly apparent after regeneration. Whereas the catalyst according to the invention achieved an initial methanol conversion of about the same order of magnitude as the still unused catalyst, comparative catalyst 3 can only be regenerated to a slight extent and the methanol conversions are markedly reduced compared with the first cycle.

1. Process for producing a phosphorus-containing catalyst, comprising the following steps:
(a) applying a phosphorus-containing compound to a zeolite,
(b) calcining the modified zeolite from step (a),
(c) treating the calcined zeolite from step (b) with an aqueous solution or water, in order to remove at least 50 wt.-% of the phosphorus-containing compound,
(d) mixing the material from step (c) with a binder,
(e) shaping the binder-zeolite mixture from step (d), and
(f) calcining the shaped material from step (e).

2. Process according to claim 1, wherein the structure of the zeolite is selected from a TON structure, MTT structure, MFI structure, MEL structure, MTW structure and EUO structure.

3. Process according to claim 1, wherein the zeolite has a silicon:aluminium ratio in the range of from 50 to 250.

4. Process according to claim 1, wherein the zeolite comprises an H-form zeolite.

5. Process according to claim 1, wherein the binder is selected from the group consisting of aluminium oxide, magnesium oxide, titanium oxide, zinc oxide, niobium oxide, zirconium oxide, silicon oxide, hydrates thereof and mixtures thereof.

6. Process according to claim 1, wherein the binder is used in an amount comprising from 5 to 60 wt.-%, relative to the total weight of zeolite and binder used.

7. Process according to claim 1, wherein the modified zeolite is calcined in step (b) at 400 to 700° C. for 5 to 15 hours.

8. Process according to claim 1, wherein the phosphorus-containing compound is applied to the zeolite in step (a) by spray-drying.

9. Process according to claim 1, wherein the aqueous solution or water of step (c) is selected from the group consisting of water, aqueous ammonium chloride, dilute hydrochloric acid, dilute acetic acid and dilute nitric acid.

10. Process according to claim 1, wherein the phosphorus-containing compound is selected from the group consisting of inorganic phosphorus-containing acids, organic phosphorus-containing acids, alkali salts, alkaline-earth salts and ammonium salts of inorganic phosphorus-containing acids and organic phosphorus-containing acids, phosphorus (V) halides, phosphorus(III) halides, phosphorus oxide halides, phosphorus(V) oxide, phosphorus(III) oxide and mixtures thereof.

11. Process according to claim 1, wherein the phosphorus-containing compound is selected from the group consisting of PY₅, PY₃, POY₃, MₓE_{z/2}H_{3-(x+z)}PO₄, MₓE_{z/2}H_{3-(x+z)}PO₃, P₂O₅ and P₄O₆, in which
Y denotes F, Cl, Br or I,
x=0, 1, 2 or 3,
z=0, 1, 2, or 3,
wherein x+z≦3,
M denotes alkali metal and/or ammonium, and
E denotes an alkaline-earth metal.

12. Process according to claim 11, wherein the phosphorus-containing compound is selected from the group consisting of H₃PO₄, (NH₄)H₂PO₄, (NH₄)₂HPO₄ and (NH₄)₃PO₄.

13. Process according to claim 1, wherein the phosphorus-containing compound comprises H₃PO₄.

14. Catalyst, obtainable by the process according to claim 1.

15. Process for producing olefins from oxygenates, comprising passing an educt gas, selected from the group consisting of methanol, dimethyl ether and mixtures thereof over the catalyst according to claim 14.

16. (canceled)

17. Process of claim 1, wherein the zeolite has a silicon:aluminum ratio in the range of 50 to 150.
